(19) United States
(12) Patent Application Publication
(10) Pub. No.: US 2023/0131785 A1
(43) Pub. Date: Apr. 27, 2023

(54) Disinfecting systems for a respirator and respirator comprising disinfecting systems

(71) Applicant: AIR Industries LLC, Metairie, LA (US)

(72) Inventors: Justin Hartenstein, Metairie, LA (US); Troy Organo, Metairie, LA (US); Alvin B Ricouard, New Orleans, LA (US)

(73) Assignee: AIR Industries LLC, Metairie, LA (US)

(21) Appl. No.: 17/907,710

(22) PCT Filed: Mar. 25, 2021
(86) PCT No.: PCT/US2021/024028

§ 371 Date: Sep. 29, 2022

Related U.S. Application Data
(60) Provisional application No. 63/006,950, filed on Apr. 8, 2020.
(60) Provisional application No. 63/015,069, filed on Apr. 24, 2020.
(60) Provisional application No. 63/032,838, filed on Jun. 1, 2020.
(60) Provisional application No. 63/139,402, filed on Jan. 20, 2021.

(51) Int. Cl. A62B 23/02; A61L 9/20

(52) U.S. Cl. CPC A62B 23/02; A61L 9/20; A61L 2209/14

(57) ABSTRACT

Described herein is a disinfecting system for a respirator. The disinfecting system may comprise a ultraviolet irradiation component configured to emit ultraviolet (UV) irradiation in a direction of a filter cartridge of the respirator. The ultraviolet irradiation component may be integrated with the filter cartridge or may be contained in a separate cover adapted to connect to the filter cartridge. The disinfecting system may also comprise a ultraviolet irradiation component configured to emit ultraviolet (UV) irradiation in a direction of an exhale port of the respirator. The ultraviolet irradiation component may be integrated with the exhale port or may be contained in a separate shield adapted to connect to the exhale port.

### CROSS REFERENCES AND PRIORITIES

This Application claims priority from U.S. Provisional Application No. 63/006,950 filed on 8 Apr. 2020, U.S. Provisional Application No. 63/015,069 filed on 24 Apr. 2020, U.S. Provisional Application No. 63/032,838 filed on 1 Jun. 2020, and U.S. Provisional Application No. 63/139,402 filed on 20 Jan. 2021, the teachings of each of which are incorporated by reference herein in their entirety.

### BACKGROUND

Existing respirators—sometimes referred to as “gas masks”—designed for chemical and biological protection are designed to remove particulates from the incoming air, but are not designed to disinfect or sanitize the particles nor are the filters designed specifically to inactivate airborne viruses, pathogens, or microorganisms. In most cases the filter cartridges of a respirator consist of a filter medium comprised of fiber material, charcoal, pleated fabric, or other filter media to remove particle material from the air that the user is inhaling. The exhaled air flows freely, unfiltered, normally through a one-way valve.

The need exists, therefore, for a respirator which disinfects and/or sanitizes the particulates from the incoming air. The need also exists for a respirator which can inactivate all or some of airborne viruses, pathogens, or microorganisms. The need further exists for a respirator which filters, disinfects, and/or sanitizes exhaled air.

### SUMMARY

It is disclosed a disinfecting system for a respirator which may comprise a cover. The cover may comprise a cover housing, a first power source, and at least one first ultraviolet irradiation component capable of emitting ultraviolet (UV) irradiation. The cover may be adapted to fit over at least a portion of a filter cartridge of the respirator. The at least one first ultraviolet irradiation component may be electrically connected to the first power source. The at least one first ultraviolet irradiation component may be configured within the cover housing to emit light in the direction of the filter cartridge when the cover is connected to the filter cartridge.

In some such embodiments, the cover housing may comprise an outer lip. The outer lip may have a plurality of protrusions extending from an interior surface thereof. The plurality of protrusions may be adapted to create a friction fit with the filter cartridge when the cover is connected to the filter cartridge.

In certain such embodiments, the first ultraviolet irradiation component may comprise at least one light emitting diode (LED) selected from the group consisting of at least one UVC light emitting diode, at least one UVA light emitting diode, and combinations thereof.

In some such embodiments, the cover may further comprise a first circuit board. The at least one light emitting diode may be electrically connected to the first circuit board. The first power source may be electrically connected to the first circuit board.

In certain such embodiments, the disinfecting system may further comprise a first switch. The first switch may be electrically connected between the first ultraviolet irradiation component and the first power source.

In some such embodiments, the first power source may be a battery.

It is further disclosed a disinfecting system for a respirator comprising a filter cartridge. The filter cartridge may comprise a cartridge housing, a cartridge filtration media located within said cartridge housing, a first power source, and at least one first ultraviolet irradiation component capable of emitting ultraviolet (UV) irradiation. The at least one first ultraviolet irradiation component may be electrically connected to the first power source. The at least one first ultraviolet irradiation component may be configured within the cartridge housing to emit light in the direction of the cartridge filtration media.

In some such embodiments, the first ultraviolet irradiation component may comprise at least one light emitting diode (LED) selected from the group consisting of at least one UVC light emitting diode, at least one UVA light emitting diode, and combinations thereof.

In certain such embodiments, the filter cartridge may further comprise a first circuit board. The at least one light emitting diode may be electrically connected to the first circuit board. The first power source may be electrically connected to the first circuit board.

In some such embodiments, the disinfecting system may further comprise a first switch. The first switch may be electrically connected between the first ultraviolet irradiation component and the first power source.

In certain such embodiments, the first power source may be a battery.

It is also disclosed a disinfecting system for a respirator comprising a shield. The shield may comprise a cap, a second power source, a shield housing, and at least one second ultraviolet irradiation component capable of emitting ultraviolet (UV) irradiation. The shield may be adapted to fit over at least a portion of an exhale port of said respirator. The at least one second ultraviolet irradiation component may be electrically connected to the second power source. The at least one second ultraviolet irradiation component may be configured within the shield housing to emit light in the direction of the exhale port when the shield is connected to the exhale port.

In some such embodiments, the second ultraviolet irradiation component may comprise at least one light emitting diode (LED) selected from the group consisting of at least one UVC light emitting diode, at least one UVA light emitting diode, and combinations thereof.

In certain such embodiments, the shield may further comprise a second circuit board. The at least one light emitting diode may be electrically connected to the second circuit board. The second power source may be electrically connected to the second circuit board.

In some such embodiments, the disinfecting system may further comprise a second switch. The second switch may be electrically connected between the second ultraviolet irradiation component and the second power source.

In certain such embodiments, the second power source may be a battery.

It is further disclosed a disinfecting system for a respirator comprising an exhale port. The exhale port may comprise an exhale port housing, an exhale port filtration media located within said exhale port housing, a second power source, and at least one second ultraviolet irradiation component capable of emitting ultraviolet (UV) irradiation. The at least one second ultraviolet irradiation component may be electrically connected to the second power source. The at least one second ultraviolet irradiation component may be configured within the exhale port housing to emit light in the direction of the exhale port filtration media.

In some such embodiments, the second ultraviolet irradiation component may comprise at least one light emitting diode (LED) selected from the group consisting of at least one UVC light emitting diode, at least one UVA light emitting diode, and combinations thereof.

In certain such embodiments, the exhale port may further comprise a second circuit board. The at least one light emitting diode may be electrically connected to the second circuit board. The second power source may be electrically connected to the second circuit board.

In some such embodiments, the disinfecting system may further comprise a second switch. The second switch may be electrically connected between the second ultraviolet irradiation component and the second power source.

In certain such embodiments, the second power source may be a battery.

It is also disclosed a respirator comprising the cover disclosed herein connected to at least one filter cartridge of the respirator, and the shield disclosed herein connected to an exhaust port of the respirator.

In some such embodiments, the cover and the shield may share a mutual power source.

It is further disclosed herein a respirator comprising the filter cartridge disclosed herein and the shield disclosed herein connected to an exhaust port of the respirator.

In some such embodiments, the filter cartridge and the shield may share a mutual power source.

It is also disclosed herein a respirator comprising the cover disclosed herein connected to at least one filter cartridge of the respirator, and the exhale port disclosed herein.

In some such embodiments, the cover and the exhale port may share a mutual power source.

It is further disclosed herein a respirator comprising the filter cartridge disclosed herein and the exhale port disclosed herein.

In some such embodiments, the filter cartridge and the exhale port may share a mutual power source.

### BRIEF DESCRIPTION OF FIGURES

FIG. 1 is an exploded perspective view of a cover.

FIG. 2 is an assembled cross section view of a cover.

FIG. 3 is an exploded perspective view of a shield.

FIG. 4 is an assembled cross section view of a shield.

FIG. 5 is a partially exploded perspective view of a respirator in use by a wearer with a cover and a shield.

FIG. 6 is an assembled perspective view of a respirator in use by a wearer with a cover and a shield.

### DETAILED DESCRIPTION

Disclosed herein are various embodiments of a disinfecting system for a respirator. The disinfecting systems are described below with reference to the Figures. As described herein and in the claims, the following numbers refer to the following structures as noted in the Figures.

10 refers to a respirator.

20 refers to a filter cartridge.

22 refers to a cartridge housing.

30 refers to an exhale port.

32 refers to an exhale port housing.

100 refers to a cover.

110 refers to a cover housing.

112 refers to an outer lip (of the cover housing).

114 refers to protrusions.

116 refers to an internal surface (of the cover housing).

120 refers to a first power source.

130 refers to a first ultraviolet irradiation component.

140 refers to a first circuit board.

150 refers to a first switch.

200 refers to a shield.

210 refers to a cap.

220 refers to a second power source.

230 refers to a shield housing.

232 refers to an internal surface (of the shield housing).

240 refers to a second ultraviolet irradiation component.

250 refers to a second circuit board.

260 refers to a second switch.

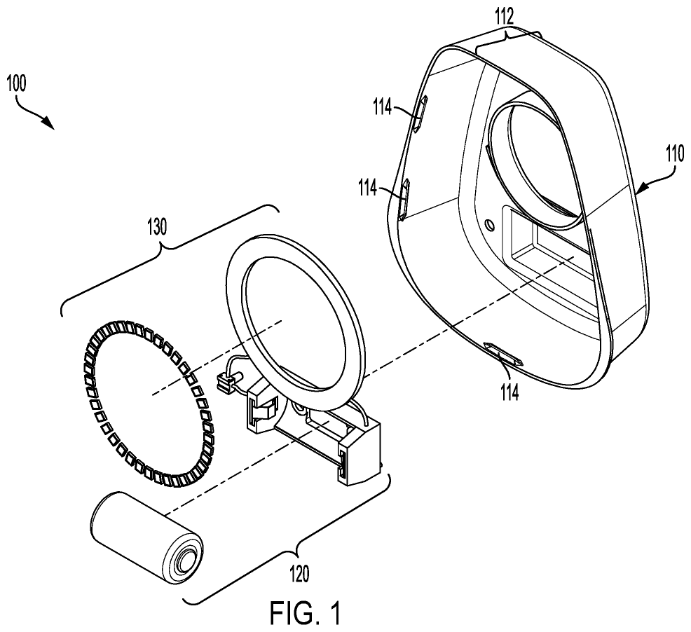

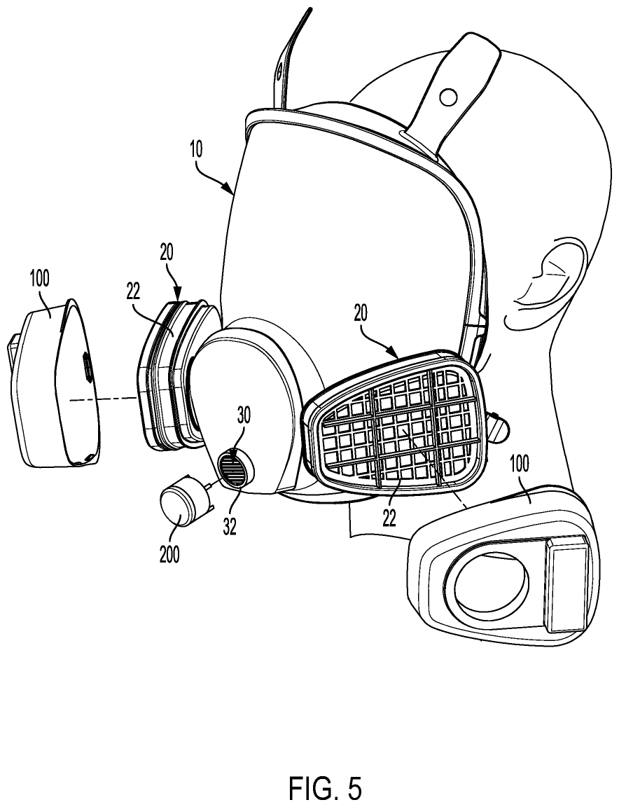

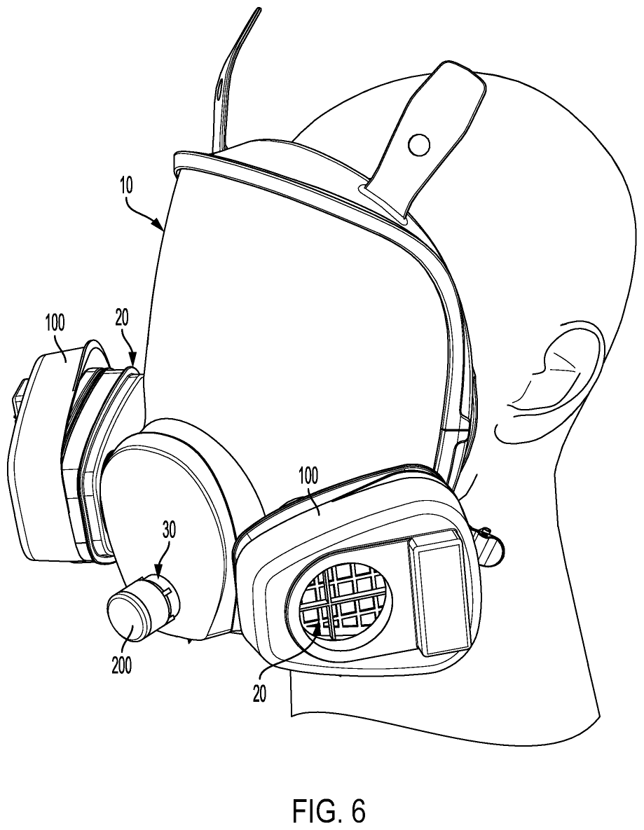

FIG. 1 depicts an exploded perspective view of one embodiment of a disinfecting system for a respirator (10 as shown in FIG. 5 and FIG. 6). As shown in FIG. 1, the disinfecting system for a respirator may comprise a cover (100). The cover may be adapted to fit over at least a portion of a filter cartridge (20 as shown in FIG. 5) of the respirator (10 as shown in FIG. 5).

As depicted in FIG. 1, the cover may comprise a cover housing (110), a first power source (120), and at least one first ultraviolet irradiation component (130). The first ultraviolet irradiation component will be capable of emitting irradiation in the form of light within the ultraviolet (UV) spectrum (also known as ultraviolet (UV) light) as described herein. As the at least one first ultraviolet irradiation component emits light—albeit in the ultraviolet (UV) spectrum—the ultraviolet irradiation component may sometimes be referred to as a lighting component with the terms “ultraviolet irradiation component” and “lighting component” intended to each refer to the same structure. This may be referred to herein as a retrofit disinfecting system for a filter cartridge.

FIG. 1 also depicts details of the cover housing (110). As shown in FIG. 1, the cover housing may have an outer lip (112). In some embodiments, the outer lip may comprise a plurality of protrusions (114) extending from an interior surface of the outer lip. The plurality of protrusions is adapted so that, when the cover is connected to the filter cartridge as shown in FIG. 5 and FIG. 6, the plurality of protrusions may create a friction fit with the filter cartridge (20 as shown in FIG. 5).

While the embodiment shown in FIG. 1 is depicted as having a friction fit which connects the cover (100) to the filter cartridge (20 as shown in FIG. 5), other connection mechanisms may be utilized. One such connection mechanism may be a threaded connection in which female threads on the interior surface of the outer lip (112) of the cover housing (110) are mated to male threads on an outer surface of the filter cartridge. In any event, it is preferred that the connection between the cover and the filter cartridge be a removable connection.

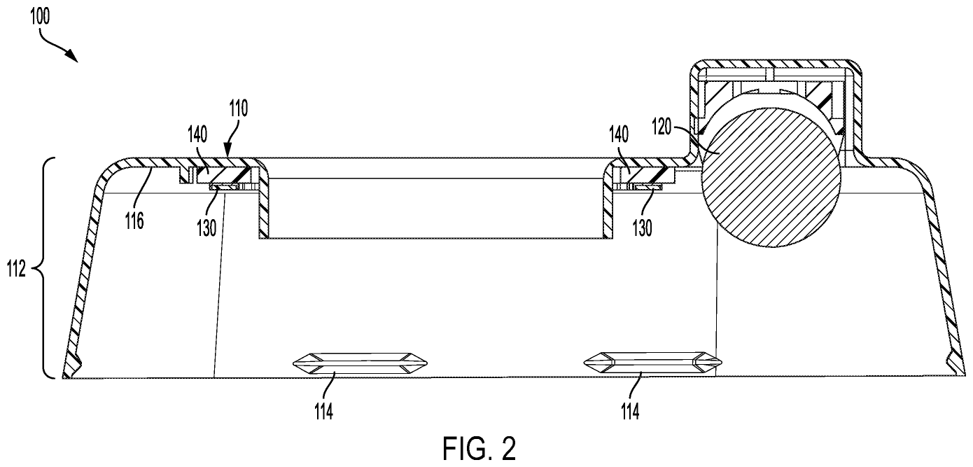

FIG. 2 depicts an assembled cross section view of the embodiment of a disinfecting system for a respirator (10). As shown in FIG. 2, once assembled, the first ultraviolet irradiation component (130) is electrically connected to the first power source. The first ultraviolet irradiation component may also be configured within the cover housing (110) to emit irradiation in the direction of the filter cartridge (20 as shown in FIG. 5) once installed on the respirator. While FIG. 2 depicts the first ultraviolet irradiation component configured to emit irradiation in the general direction of the upstream side of the filter cartridge, other configurations may exist in which the first ultraviolet irradiation component is configured to emit irradiation in the general direction of the downstream side of the filter cartridge. In still other embodiments, the first ultraviolet irradiation component may be configured to emit irradiation in the general direction of both the upstream side and the downstream side of the filter cartridge.

In some embodiments, the first ultraviolet irradiation component may comprise at least one light emitting diode—also referred to herein as an LED. The light emitting diode(s) may be designed to create a wavelength of light having disinfecting characteristics. This type of disinfecting light is commonly known as ultraviolet germicidal irradiation (UVGI). UVGI is a disinfection method that uses short-wavelength ultraviolet A (UVA), ultraviolet B (UVB), and/or ultraviolet C (UVC) light to kill or inactivate microorganisms by destroying nucleic acids which, in turn, disrupts their DNA, rendering them inactive by leaving these cells unable to perform vital cellular functions.

The UVGI light produced by the light emitting diode(s) may aid in sterilization of air being inhaled to the respirator as well as neutralizing contaminants trapped in the filter cartridge itself. The irradiation of the UVGI light utilizes photons to disinfect the inhaled air. UVGI light damages a pathogen's DNA or RNA, which prevents it from replicating and infecting the body if inhaled. In addition to disinfecting inhaled air, the UVGI light emitting diode(s) may also sterilize the filter material of the filter cartridge itself.

The inhaled air may be exposed to the ultraviolet irradiation from the UVGI light emitting diode(s). UV light is electromagnetic radiation with wavelengths shorter than visible light, but longer than X-rays. UV can be separated into various ranges, with short-wavelength UVC generally considered to be “germicidal UV”. Wavelengths between about 200 nm and 300 nm are strongly absorbed by nucleic acids. The absorbed energy can result in defects including pyrimidine dimers. These dimers can prevent replication or prevent the expression of necessary proteins, resulting in the death or inactivation of the organism. However, light emitting diodes which emit UV light in a range selected from the group consisting of between 100 to 400 nm wavelengths, between 100 and 300 nm wavelengths, between 200 and 400 nm wavelengths, between 200 and 300 nm wavelengths, and between 300 and 400 nm wavelengths may also be utilized. The light emitting diode of the first ultraviolet irradiation component may comprise at least one light emitting diode selected from the group consisting of at least one UVC light emitting diode, at least one UVA light emitting diode, and combinations thereof.

In certain embodiments, when the first ultraviolet irradiation component (130) comprises a light emitting diode, the first ultraviolet irradiation component may further comprise a first circuit board (140). In such embodiments, at least one of the light emitting diode(s) may be electrically connected to the first circuit board. Preferably, each of the light emitting diode(s) is electrically connected to the first circuit board. The first circuit board, in turn, will be electrically connected to the first power source (120).

In some embodiments, the disinfecting system may also comprise a first switch. When present, the first switch may be electrically connected between the first ultraviolet irradiation component (130) and the first power source (120). The first switch may be configured to allow a user to turn the first ultraviolet irradiation component on and off by changing the switch from an on position in which the switch closes the electrical circuit between the first power source and the first ultraviolet irradiation component allowing electrical current from the power source to flow to the first ultraviolet irradiation component, to an off position in which the first switch opens the electrical circuit between the first power source and the first ultraviolet irradiation component preventing electrical current from the first power source from flowing to the first ultraviolet irradiation component.

Instead of, or in addition to, the first switch—some embodiments may include a first sensor which is electrically connected between the first ultraviolet irradiation component (130) and the first power source (120). The first sensor may be configured to automatically turn the first ultraviolet irradiation component on and off upon detection of a specific condition—such as detection of a breathing from a user wearing the respirator. When the sensor detects the presence of the specific condition, the sensor closes the electrical circuit between the first power source and the first ultraviolet irradiation component allowing electrical current from the power source to flow to the first ultraviolet irradiation component. When the sensor detects the absence of the specific condition, the sensor opens the electrical circuit between the first power source and the first ultraviolet irradiation component preventing electrical current from the power source from flowing to the first ultraviolet irradiation component. Non-limiting examples of such sensors may include an air flow sensor or an air pressure sensor.

In some embodiments, the first switch may be integrally connected to the first power source (120) within a first power source housing. In other embodiments, the first switch may be a stand-alone switch attached to another component of the cover (100) or the respirator (10 as shown in FIG. 5 and FIG. 6). In such embodiments, there may be two separate wires—one of which electrically connects the first power source to the first switch while the other electrically connects the first switch to the first ultraviolet irradiation component (130).

The preferred first power source is a battery—preferably a rechargeable battery. Examples of such batteries include lithium-ion batteries, lithium-ion polymer batteries, nickel-cadmium batteries, and nickel-metal hydride batteries. In some embodiments, the power source may also comprise a recharging mechanism—such as a solar cell, a wind energy generator, and/or a breath-driven turbine—electrically connected to the battery.

Another example of a recharging mechanism may include an electrical connection which can be plugged into a standard wall outlet via a cable.

FIG. 2 shows an assembled cross-section view of the disinfecting system shown in FIG. 1. As shown in FIG. 2, once assembled, the first ultraviolet irradiation component (130) may be connected to the cover housing (110) at an internal surface (116) of the cover housing. In some embodiments, such as that shown in FIG. 2, the first ultraviolet irradiation component may be in the form of at least one light-emitting diode (LED), in which case the cover may further comprise a first circuit board (140). In embodiments where the cover comprises a first circuit board, the at least one light-emitting diode may be electrically connected to the first circuit board, and the first power source (120) may be electrically connected to the first circuit board.

FIG. 2 also illustrates the protrusions (114). As shown in FIG. 2, each protrusion is located on an interior surface of the outer lip (112). These protrusions may the sized and located to correspond with surface(s) of the filter cartridge (20 as shown in FIG. 5) such that the protrusions allow for a friction fit of the cover to the filter cartridge when the cover is connected to the filter cartridge.

While FIG. 1 and FIG. 2 show the disinfecting system for a respirator (10) comprising a cover (100) (also known as a retrofit disinfecting system for a filter cartridge), the cover is not considered a necessary element. In certain embodiments, the disinfecting system may comprise a first power source and at least one first ultraviolet irradiation component configured within the cartridge housing of an existing filter cartridge (20 as shown in FIG. 5). This may also be referred to herein as an integrated disinfecting system for a filter cartridge.

In such embodiments, the at least one first ultraviolet irradiation component may be electrically connected to the first power source (with or without a first switch) in the same manner that the first power source is electrically connected to the first ultraviolet irradiation component in the embodiment shown in FIG. 1 and FIG. 2. The first power source may be of any type disclosed herein with reference to the embodiment shown in FIG. 1 and FIG. 2. The at least one first ultraviolet irradiation component may be configured within the cartridge housing to emit irradiation in the direction of a cartridge filtration media which is also located within the cartridge housing. This may involve configuring the at least one first ultraviolet irradiation component to emit irradiation in the general direction of the upstream side of the filtration media, the downstream side of the filtration media, or both the upstream and downstream sides of the filtration media.

The at least one first ultraviolet irradiation component in such embodiments may include any of the ultraviolet irradiation components disclosed herein with reference to the embodiment shown in FIG. 1 and FIG. 2. Specific preferred ultraviolet irradiation components include at least one light emitting diode (LED) selected from the group consisting of at least one UVC light emitting diode, at least one UVA light emitting diode, and combinations thereof. Preferably, when the at least one first ultraviolet irradiation component include a light emitting diode (LED), the filter cartridge will further comprise a first circuit board as described herein with reference to the embodiment shown in FIG. 1 and FIG. 2.

In some embodiments, all or a portion of the irradiation emitted from the first ultraviolet irradiation component (130) may be directed towards a reflective surface. The reflective surface may be a surface of the cover (100), a surface of the filter cartridge (20), or an additional surface attached to the cover or the filter cartridge. A preferred reflective surface is a surface which has been treated with evaporated aluminum. By directing all or a portion of the irradiation emitted from the first ultraviolet irradiation component towards a reflective surface, the light may reflect off of the surface thereby increasing the dispersion of the light. This can increase the surface area of the filter cartridge which is exposed to the emitted irradiation, and also increase the duration of time which particles in the air are exposed to the emitted irradiation—both of which are thought to improve the ability of the light to neutralize bacterial and/or viral particles.

In certain embodiments, the cover (100) and/or the filter cartridge (20) may include a circuitous pathway. A circuitous pathway as used herein and in the claims describes a tube, passage, conduit, or the like which increases the length of the pathway that inbound air is directed through when passing through the filter cartridge. The circuitous pathway may take many forms with a serpentine pathway and a spiral pathway being considered non-limiting examples of preferred pathways. Preferably all or a majority of the inner surface of the circuitous pathway will be exposed to the irradiation emitted from the first ultraviolet irradiation component with at least 51% of the inner surface of the circuitous pathway being exposed to the irradiation emitted from the first ultraviolet irradiation component being preferred, at least 75% being more preferred, at least 90% being still more preferred, and at least 99% being most preferred. The circuitous pathway is preferably an integral component of the cover and/or the filter cartridge such as by injection molding the circuitous path as part of the cover and/or the filter cartridge. However, embodiments may exist in which the circuitous pathway is a separate component which is connected between an inlet of the cover and an inlet of the filter cartridge.

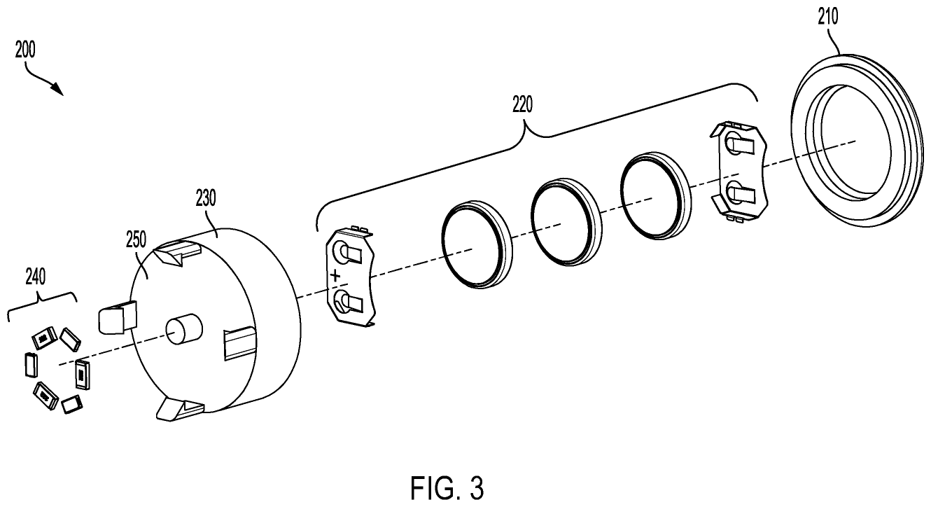

While the embodiments described above are useful as disinfecting systems for the inhale components of a respirator, they do not address issues associated with the exhale components of a respirator. Accordingly, instead of or in addition to the embodiments described above, one embodiment includes a disinfecting system for a respirator (10) as shown in FIG. 3.

FIG. 3 shows the disinfecting system for a respirator (10) comprising a shield (200) adapted to fit over at least a portion of an exhale port (30 as shown in FIG. 5) of a respirator. As shown in FIG. 3, the shield may comprise a cap (210), a second power source (220), a shield housing (230), and at least one second ultraviolet irradiation component (240). The second ultraviolet irradiation component will be capable of emitting irradiation in the form of light within the ultraviolet (UV) spectrum (also known as ultraviolet (UV) light) as described herein. As the at least one second ultraviolet irradiation component emits light—albeit in the ultraviolet (UV) spectrum—the ultraviolet irradiation component may sometimes be referred to as a lighting component with the terms “ultraviolet irradiation component” and “lighting component” intended to each refer to the same structure. This may be referred to herein as a retrofit disinfecting system for an exhale port.

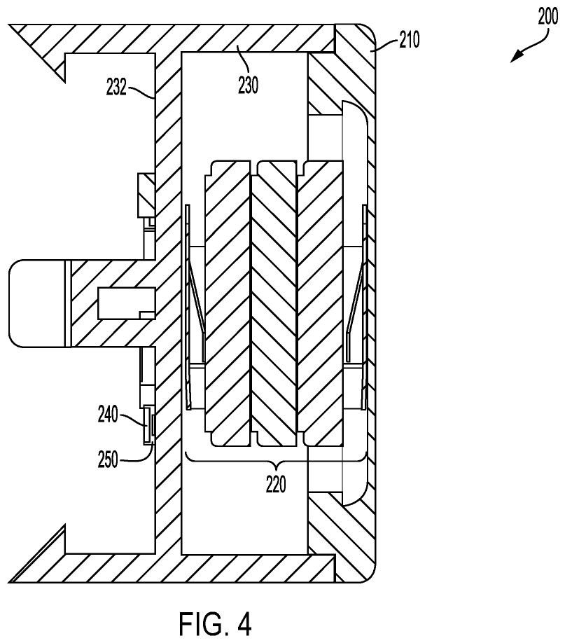

FIG. 3 also shows details of the shield (200). As shown in FIG. 3 the cap (210) may be configured to connect to the shield housing (230) which holds the second power source (220) in place between the cap and the shield housing as shown in FIG. 4. The shield housing may also comprise at least one fastening mechanism configured to connect the shield to at least a portion of an exhale port (30 as shown in FIG. 5) of the respirator (10).

The at least one second ultraviolet irradiation component (240) may be configured within the shield housing (230) to emit irradiation in the direction of the exhale port when the shield (200) is connected to the exhale port (30) as shown in FIG. 5 and FIG. 6. While FIG. 3 depicts the second ultraviolet irradiation component configured to emit irradiation in the general direction of the downstream side of the exhale port, other configurations may exist in which the second ultraviolet irradiation component is configured to emit irradiation in the general direction of the upstream side of the exhale port. In still other embodiments, the second ultraviolet irradiation component may be configured to emit irradiation in the general direction of both the upstream side and the downstream side of the exhale port.

In some embodiments, the second ultraviolet irradiation component may comprise at least one light emitting diode—also referred to herein as an LED. The light emitting diode(s) may be designed to create a wavelength of light having disinfecting characteristics. This type of disinfecting light is commonly known as ultraviolet germicidal irradiation (UVGI). UVGI is a disinfection method that uses short-wavelength ultraviolet A (UVA), ultraviolet B (UVB), and/or ultraviolet C (UVC) light to kill or inactivate microorganisms by destroying nucleic acids which, in turn, disrupts their DNA, rendering them inactive by leaving these cells unable to perform vital cellular functions.

The UVGI light produced by the light emitting diode(s) may aid in sterilization of air being exhaled from the respirator as well as neutralizing contaminants trapped in the exhale port itself. The irradiation of the UVGI light utilizes photons to disinfect the exhaled air. UVGI light damages a pathogen's DNA or RNA, which prevents it from replicating and infecting the body if inhaled. In addition to disinfecting exhaled air, the UVGI light emitting diode(s) may also sterilize the filter material of the exhale port itself.

The exhaled air may be exposed to the ultraviolet irradiation from the UVGI light emitting diode(s). UV light is electromagnetic radiation with wavelengths shorter than visible light, but longer than X-rays. UV can be separated into various ranges, with short-wavelength UVC generally considered to be “germicidal UV”. Wavelengths between about 200 nm and 300 nm are strongly absorbed by nucleic acids. The absorbed energy can result in defects including pyrimidine dimers. These dimers can prevent replication or prevent the expression of necessary proteins, resulting in the death or inactivation of the organism. However, light emitting diodes which emit UV light in a range selected from the group consisting of between 100 to 400 nm wavelengths, between 100 and 300 nm wavelengths, between 200 and 400 nm wavelengths, between 200 and 300 nm wavelengths, and between 300 and 400 nm wavelengths may also be utilized. The light emitting diode of the second ultraviolet irradiation component may comprise at least one light emitting diode selected from the group consisting of at least one UVC light emitting diode, at least one UVA light emitting diode, and combinations thereof.

In certain embodiments, when the second ultraviolet irradiation component (240) comprises a light emitting diode, the second ultraviolet irradiation component may further comprise a second circuit board (250). In such embodiments, at least one of the light emitting diode(s) may be electrically connected to the second circuit board. Preferably, each of the light emitting diode(s) is electrically connected to the second circuit board. The second circuit board, in turn, will be electrically connected to the second power source (220).

In some embodiments, the disinfecting system may also comprise a second switch. When present, the second switch may be electrically connected between the second ultraviolet irradiation component (240) and the second power source (220). The second switch may be configured to allow a user to turn the second ultraviolet irradiation component on and off by changing the switch from an on position in which the switch closes the electrical circuit between the second power source and the second ultraviolet irradiation component allowing electrical current from the power source to flow to the second ultraviolet irradiation component, to an off position in which the second switch opens the electrical circuit between the second power source and the second ultraviolet irradiation component preventing electrical current from the second power source from flowing to the second ultraviolet irradiation component.

Instead of, or in addition to, the second switch—some embodiments may include a second sensor which is electrically connected between the second ultraviolet irradiation component (240) and the first power source (220). The second sensor may be configured to automatically turn the second ultraviolet irradiation component on and off upon detection of a specific condition—such as detection of a breathing from a user wearing the respirator. When the sensor detects the presence of the specific condition, the sensor closes the electrical circuit between the second power source and the second ultraviolet irradiation component allowing electrical current from the power source to flow to the second ultraviolet irradiation component. When the sensor detects the absence of the specific condition, the sensor opens the electrical circuit between the second power source and the second ultraviolet irradiation component preventing electrical current from the power source from flowing to the second ultraviolet irradiation component. Non-limiting examples of such sensors may include an air flow sensor or an air pressure sensor.

In some embodiments, the second switch may be integrally connected to the second power source (220) within a second power source housing. In other embodiments, the second switch may be a stand-alone switch attached to another component of the shield (200) or the respirator (10 as shown in FIG. 5 and FIG. 6). In such embodiments, there may be two separate wires—one of which electrically connects the second power source to the second switch while the other electrically connects the second switch to the second ultraviolet irradiation component (240).

In certain embodiments, the disinfecting system for the inhale components of the respirator may share a switch with the disinfecting system for exhale components of the respirator. That is to say that there may be a common switch which controls the flow of electrical current to both the first ultraviolet irradiation component and the second ultraviolet irradiation component. In such embodiments, there may be a first wire connecting the first power source to the common switch, a second wire connecting the second power source to the common switch, a third wire connecting the common switch to the first ultraviolet irradiation component, and a fourth wire connecting the common switch to the second ultraviolet irradiation component. When the common switch is turned to an on position, circuits are closed between the first power source and the first ultraviolet irradiation component, and the second power source and the second ultraviolet irradiation component respectively allowing electrical current to flow to each of the first and second ultraviolet irradiation components. Conversely, when the common switch is turned to an off position, circuits are opened between the first power source and the first ultraviolet irradiation component, and the second power source and the second ultraviolet irradiation component respectively stopping the flow of electrical current to each of the first and second ultraviolet irradiation components.

The preferred second power source is a battery—preferably a rechargeable battery. Examples of which include lithium-ion batteries, lithium-ion polymer batteries, nickel-cadmium batteries, and nickel-metal hydride batteries. In some embodiments, the second power source may also comprise a recharging mechanism—such as a solar cell, a wind energy generator, and/or a breath-driven turbine—electrically connected to the battery. Another example of a recharging mechanism may include an electrical connection which can be plugged into a standard wall outlet via a cable.

FIG. 4 shows an assembled cross-section view of the disinfecting system shown in FIG. 3. As shown in FIG. 4, once assembled, the second ultraviolet irradiation component (240) may be connected to the shield housing (230) at an internal surface (232) of the shield housing. In some embodiments, such as that shown in FIG. 4, the first ultraviolet irradiation component may be in the form of at least one light-emitting diode (LED), in which case the shield may further comprise a second circuit board (250). In embodiments where the shield comprises a second circuit board, the at least one light-emitting diode may be electrically connected to the second circuit board, and the second power source (220) may be electrically connected to the second circuit board.

FIG. 4 also illustrates the second power source (220), which may comprise a plurality of batteries. As shown in FIG. 4, the plurality of batteries may be held in place between the cap (210) and the shield housing (230).

While FIG. 3 and FIG. 4 show the disinfecting system for a respirator (10) comprising a shield (200) (also known as a retrofit disinfecting system for an exhale port), the shield is not considered a necessary element. In certain embodiments, the disinfecting system may comprise a second power source and at least one second ultraviolet irradiation component configured within an existing exhale port (30 as shown in FIG. 5). This may be referred to herein as an exhale port having an integrated disinfecting system.

In embodiments of an exhale port having an integrated disinfecting system the at least one second ultraviolet irradiation component may be electrically connected to the second power source (with or without a second switch) in the same manner that the second power source is electrically connected to the second ultraviolet irradiation component in the embodiment shown in FIG. 3 and FIG. 4. The second power source may be of any type disclosed herein with reference to the embodiment shown in FIG. 3 and FIG. 4. The at least one second ultraviolet irradiation component may be configured within the exhale port housing to emit irradiation in the direction of an exhale port filtration media which is also located within the exhale port housing. This may involve configuring the at least one second ultraviolet irradiation component to emit irradiation in the general direction of the upstream side of the filtration media, the downstream side of the filtration media, or both the upstream and downstream sides of the filtration media.

The at least one second ultraviolet irradiation component in such embodiments may include any of the ultraviolet irradiation components disclosed herein with reference to the embodiment shown in FIG. 3 and FIG. 4. Specific preferred ultraviolet irradiation components include at least one light emitting diode (LED) selected from the group consisting of at least one UVC light emitting diode, at least one UVA light emitting diode, and combinations thereof. Preferably, when the at least one second ultraviolet irradiation component include a light emitting diode (LED), the exhale port will further comprise a second circuit board as described herein with reference to the embodiment shown in FIG. 3 and FIG. 4.

In some embodiments, all or a portion of the irradiation emitted from the second ultraviolet irradiation component (240) may be directed towards a reflective surface. The reflective surface may be a surface of the shield (200), a surface of the exhale port (30), or an additional surface attached to the shield or the exhale port. A preferred reflective surface is a surface which has been treated with evaporated aluminum. By directing all or a portion of the irradiation emitted from the second ultraviolet irradiation component towards a reflective surface, the light may reflect off of the surface thereby increasing the dispersion of the light. This can increase the surface area of the exhale port which is exposed to the emitted irradiation, and also increase the duration of time which particles in the air are exposed to the emitted irradiation—both of which are thought to improve the ability of the light to neutralize bacterial and/or viral particles.

In certain embodiments, the shield (200) and/or the exhale port (30) may include a circuitous pathway. A circuitous pathway as used herein and in the claims describes a tube, passage, conduit, or the like which increases the length of the pathway that outbound air is directed through when passing through the exhale port. The circuitous pathway may take many forms with a serpentine pathway and a spiral pathway being considered non-limiting examples of preferred pathways. Preferably all or a majority of the inner surface of the circuitous pathway will be exposed to the irradiation emitted from the second ultraviolet irradiation component with at least 51% of the inner surface of the circuitous pathway being exposed to the irradiation emitted from the second ultraviolet irradiation component being preferred, at least 75% being more preferred, at least 90% being still more preferred, and at least 99% being most preferred. The circuitous pathway is preferably an integral component of the shield and/or the exhale port such as by injection molding the circuitous path as part of the shield and/or the exhale port. However, embodiments may exist in which the circuitous pathway is a separate component which is connected between an inlet of the shield and an inlet of the exhale port.

FIG. 5 and FIG. 6 show the retrofit disinfecting system for a filter cartridge and the retrofit disinfecting system for an exhale port connected to a respirator (10). As shown in FIG. 5, the respirator may comprise at least one filter cartridge (20). However, many embodiments will comprise two filter cartridges. Each filter cartridge will comprise a cartridge housing (22) and a cartridge filtration media located within the cartridge housing. The respirator may further comprise at least one exhale port (30). Each exhale port will comprise an exhale port housing (32) and an exhale port filtration media located within the exhale port housing.

Once assembled, as shown in FIG. 6, a retrofit disinfecting system for a filter cartridge is connected to at least one—and preferably each—of the filter cartridge(s). This occurs by connecting the cover (100) to the filter cartridge housing as described herein. Similarly, a retrofit disinfecting system for an exhale port is connected to at least one—and preferably each—of the exhale port(s). This occurs by connecting the shield (200) to the exhale port housing as described herein.

While FIG. 5 and FIG. 6 show embodiments having a retrofit disinfecting system for a filter cartridge and a retrofit disinfecting system for an exhale port, other embodiments may exist. For example, one embodiment may include a retrofit disinfecting system for a filter cartridge and a retrofit disinfecting system for an exhale port. Another embodiment may include an integrated disinfecting system for a filter cartridge and a retrofit disinfecting system for an exhale port. Yet another embodiment may include a retrofit disinfecting system for a filter cartridge and an integrated disinfecting system for an exhale port. Still another embodiment may include an integrated disinfecting system for a filter cartridge and an integrated disinfecting system for an exhale port.

In embodiments having a disinfecting system for a filter cartridge and a disinfecting system for an exhale port, the disinfecting systems may share a mutual power source. That is to say that a single power source provides electrical current to the first ultraviolet irradiation component of the disinfecting system for a filter cartridge and the second ultraviolet irradiation component of the disinfecting system for an exhale port. This may be achieved by having a first wire electrically connected between the mutual power source and the first ultraviolet irradiation component (with or without a switch) and a second wire electrically connected between the mutual power source and the second ultraviolet irradiation component.

The disinfecting systems disclosed herein may assist in disinfecting and/or sanitizing the air incoming to the respirator due to the ultraviolet irradiation component generating germicidal ultraviolet light in and/or around the filter cartridge. Similarly, the disinfecting systems disclosed herein may also assist in disinfecting and/or sanitizing the air outgoing from the respirator due to the ultraviolet irradiation component generating germicidal ultraviolet light in and/or around the exhale port.

In addition, the disinfecting system disclosed herein may assist in disinfecting and/or sanitizing the filtration media of the filter cartridge by orienting the ultraviolet irradiation component to generate germicidal ultraviolet light in the direction of the filter cartridge. This can help to ensure that any viral or bacterial particles trapped in the filtration media are rendered inactive should they escape from the filtration media.

Similarly, the disinfecting systems disclosed herein may assist in disinfecting and/or sanitizing the components of the exhale port by orienting the ultraviolet irradiation component to generate germicidal ultraviolet light in the direction of the exhale port. This can help to ensure that any viral or bacterial particles trapped in the components of the exhale port are rendered inactive should they escape from the exhale port.

1. A disinfecting system for a respirator (10), comprising a cover (100) adapted to fit over at least a portion of a filter cartridge (20) of said respirator, said cover comprising a cover housing (110), a first power source (120), and at least one first ultraviolet irradiation component (130) capable of emitting ultraviolet (UV) irradiation; wherein the at least one first ultraviolet irradiation component is electrically connected to the first power source, and the at least one first ultraviolet irradiation component is configured within the cover housing to emit irradiation in the direction of the filter cartridge when the cover is connected to the filter cartridge.

2. The disinfecting system of claim 1, wherein the cover housing comprises an outer lip (112) having a plurality of protrusions (114) extending from an interior surface thereof, wherein the plurality of protrusions are adapted to create a friction fit with the filter cartridge when the cover is connected to the filter cartridge.

3. The disinfecting system of claim 1, wherein the first ultraviolet irradiation component comprises at least one light emitting diode (LED) selected from the group consisting of at least one UVC light emitting diode, at least one UVA light emitting diode, and combinations thereof.

4. The disinfecting system of claim 3, wherein the cover further comprises a first circuit board (140), the at least one light emitting diode is electrically connected to the first circuit board, and the first power source is electrically connected to the first circuit board.

5. The disinfecting system of claim 1, further comprising a first switch (150) electrically connected between the first ultraviolet irradiation component and the first power source.

6. The disinfecting system of claim 1, wherein the first power source is a battery.

7. A disinfecting system for a respirator (10), comprising a filter cartridge (20), said filter cartridge comprising a cartridge housing (22), a cartridge filtration media located within said cartridge housing, a first power source (120), and at least one first ultraviolet irradiation component (130) capable of emitting ultraviolet (UV) irradiation; wherein the at least one first ultraviolet irradiation component is electrically connected to the first power source, and the at least one first ultraviolet irradiation component is configured within the cartridge housing to emit irradiation in the direction of the cartridge filtration media.

8. The disinfecting system of claim 7, wherein the first ultraviolet irradiation component comprises at least one light emitting diode (LED) selected from the group consisting of at least one UVC light emitting diode, at least one UVA light emitting diode, and combinations thereof.

9. The disinfecting system of claim 8, wherein the filter cartridge further comprises a first circuit board (140), the at least one light emitting diode is electrically connected to the first circuit board, and the first power source is electrically connected to the first circuit board.

10. The disinfecting system of claim 7, further comprising a first switch (150) electrically connected between the first ultraviolet irradiation component and the first power source.

11. The disinfecting system of claim 7, wherein the first power source is a battery.

12. A disinfecting system for a respirator (10), comprising a shield (200) adapted to fit over at least a portion of an exhale port (30) of said respirator, said shield comprising a cap (210), a second power source (220), a shield housing (230), and at least one second ultraviolet irradiation component (240) capable of emitting ultraviolet (UV) irradiation; wherein the at least one second ultraviolet irradiation component is electrically connected to the second power source, and the at least one second ultraviolet irradiation component is configured within the shield housing to emit irradiation in the direction of the exhale port when the shield is connected to the exhale port.

13. The disinfecting system of claim 12, wherein the second ultraviolet irradiation component comprises at least one light emitting diode (LED) selected from the group consisting of at least one UVC light emitting diode, at least one UVA light emitting diode, and combinations thereof.

14. The disinfecting system of claim 13, wherein the shield further comprises a second circuit board (250), the at least one light emitting diode is electrically connected to the second circuit board, and the second power source is electrically connected to the second circuit board.

15. The disinfecting system of claim 12, further comprising a second switch (260) electrically connected between the second ultraviolet irradiation component and the second power source.

16. The disinfecting system of claim 12, wherein the second power source is a battery.

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. The disinfecting system of claim 2, wherein the first ultraviolet irradiation component comprises at least one light emitting diode (LED) selected from the group consisting of at least one UVC light emitting diode, at least one UVA light emitting diode, and combinations thereof.
